(19) United States
(12) Patent Application Publication
(10) Pub. No.: US 2007/0260312 A1
(43) Pub. Date: Nov. 8, 2007

(54) Subcutaneous Intramuscular Mounting for a Rigid Transcutaneous Implant

(76) Inventor: Hans Grundei, Lubeck (DE)

Correspondence Address: FLASTER/GREENBERG P.C.;8 PENN CENTER, 1628 JOHN F. KENNEDY BLVD., 15TH FLOOR, PHILADELPHIA, PA, 19103, US

(21) Appl. No.: 10/594,657

(22) PCT Filed: Aug. 23, 2005
(86) PCT No.: PCT/EP05/09075

§ 371 Date: May 29, 2007

(30) Foreign Application Priority Data
Nov. 11, 2004 (DE) 10 2004 055 623.7

(51) Int. Cl. A61F 2/28

(52) U.S. Cl. 623/16.11

(57) ABSTRACT

A subcutaneous, intramuscular mounting for a rigid transcutaneous implant is disclosed, which may be fixed intracorporally in a bone stump, comprising a spacer (3) between the implant and an extracorporal coupling device (4) which may be coupled thereto, whereby the spacer (3) is embodied as a rigid bush (5) with a coupling element (6), sealed in the intracorporal direction, to which the extracorporal coupling device may be coupled. According to the invention, the bush (5) widens out significantly from the end (12) thereof facing the extracorporal direction to the end (13) thereof facing the intracorporal direction and comprises a smooth surface such that arthrogenic material can not grow into the surface thereof and as a result of a lack of frictional effects also causes hardly any inflammation.

### BACKGROUND OF THE INVENTION

The invention concerns a subcutaneous, intramuscular mounting for a rigid transcutaneous implant, which may be fixed intracorporally into a bone stump, and which comprises a spacer between the implant and an extracorporal coupling device, which may be coupled thereto. The coupling element is embodied as a rigid bush, sealed in the intracorporal direction, to which the extracorporeal coupling device may be coupled.

A mounting of this type is known from DE 102 47 397 B3. This comprises a tube, applied to the outer wall of the bush and constructed of a flexible material, and metal wool applied to the flexible tube. By this means, the objective is pursued, that protection against [microbial] contamination of the passage point of the implant and the bordering areas of the femoral stump is greatly increased, and that an accidental removal of the microbial barrier is avoided. This can occur when using a mounting according to DE 100 40 590 A1, for example, when cleaning the passage point of the implant through, for example, the femoral stump, by an accidental piercing of the flexible material (usually silicon in most cases) with a cannula (hypodermic needle).

The increase in protection against contamination in the mounting according to this patent [DE 102 47 397 B3] is significantly increased due to the following: surrounding tissue granulates in to the metal wool. This functions clearly in slender and muscular patients. In a different patient group, namely the adipose patients, difficulties with this can occur, because, in this case, the implant is surrounded by a lot of fatty tissue and little muscle. Fatty tissue, however, shows almost no granulation in to the metal wool. In these patients, an irritation of the tissue surrounding the implant can occur; which does not lead, however, to an infiltrative growth of the metal wool. The constant friction poses a danger of contamination in this patient group.

### BRIEF SUMMARY OF THE INVENTION

Against this background, it is the object of the present invention to further fashion a subcutaneous, intramuscular mounting, so that the protection against contamination of the passage point of the implant and the bordering areas of the femoral stump is again significantly increased, above all in adipose patients.

This object is achieved in the following manner: the bush widens out significantly from the extracorporally oriented side to the intracorporally oriented side and comprises a smooth surface. The soft tissues surrounding the spacer, specifically the bush, atrophy onto the bush, and significantly more so in the area of the bush facing the intracorporal direction than in the part facing the extracorporal direction, due to the special design of the bush. This adds up to a constantly increasing seal from the part of the bush facing in the extracorporal direction to the part facing in the intracorporal direction. This can also be expected from surrounding fatty tissue, so that adipose patients can also be optimally cared for.

The widening of the bush occurs at a ratio from 1:1.2 to 1:2, if the length of the base edge of the bush at the extracorporally oriented end is set as 1. By this means, a sufficient “champagne cork effect” of the bush in relation to the seal is achieved.

According to an especially preferred exemplary embodiment, the surface of the bush has an antibacterial effect. For this, the surface, according to the exemplary embodiment, can be plated with silver or titanium.

According to an especially preferred exemplary embodiment, the mounting comprises an adaptation tube, which reaches into the inside of the bush, is seated in an interference fit, and is removable. The coupling device can be coupled into the adaptation tube. The adaptation tube has an antibacterial effect, at least on its outer wall. Through the use of the adaptation tube, a particularly high germicidal barrier is achieved.

If the antibacterial effect of the outer wall decreases over time, after loosening the interference fit, it [the old adaptation tube] can be replaced by a new, “fresh” adaptation tube. In order to accomplish this, the extracorporal coupling device merely has to be uncoupled from the spacer and the adaptation tube pulled out.

In a preferred, further formation, the adaptation tube is constructed of solid silver. The antibacterial effect of silver is generally known.

According to another exemplary embodiment, the adaptation tube is constructed of a material, whose outer wall is plated with silver. This exemplary embodiment is more cost efficient that the previously mentioned. For example, a cobalt-chromium-molybdenum alloy could find use as a base material.

Alternately, the adaptation tube can be constructed of a material that is coated on the outer wall with hydroxylapatite, calcium phosphate, titanium, or plasma titanium spray. The previously mentioned coating materials have the characteristic, that skin and connective tissue adsorbs on the outer wall, and thus on to the length of the adaptation tube, forming an additional germicidal effect. Nevertheless, the connective tissue, that will eventually be present, does not grow into the surface of the tube, but rather merely adsorbs on it, so that the removeability of the adaptation tube for the purpose of replacement remains guaranteed.

Alternately, the adaptation tube can be constructed of polyurethane in still another exemplary embodiment. Polyurethane is used in medical technology as an antibacterial material.

According to a particular exemplary embodiment, it is envisioned, that the adaptation tube comprises such a length, that it is situated with its distal front edge on a shoulder, which shoulder is formed on the extracorporal coupling device, when the extracorporal coupling device is coupled to the coupling element. By this means, a clean transition from the extracorporal coupling device to the spacer is achieved, and fouling or contamination of the inside of the adaptation tube is largely avoided.

### BRIEF DESCRIPTION OF THE SEVERAL VIEWS OF THE DRAWING

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 is a schematic, partial cross-sectional view of a spacer of an embodiment of the invention having a coupled adaptation tube and extracorporal coupling device.

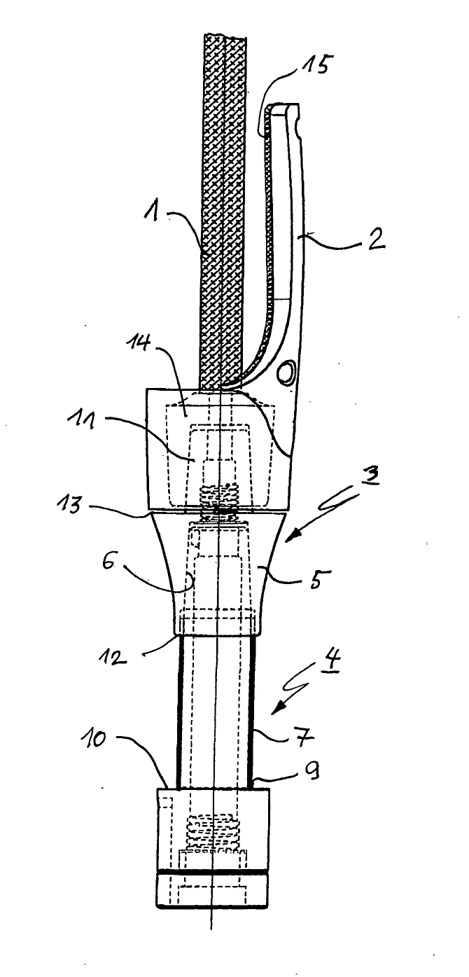

### DETAILED DESCRIPTION OF THE INVENTION

The invention will be more closely exemplified by means of the single schematic figure. This shows in cross-section the spacer with the coupled adaptation tube as well as the extracorporal coupling device.

The spacer 3 is embodied as a bush 5 and located between the transcutaneous implant 1, with which the spacer can be connected via a plug cone 11, and an extracorporal coupling device 4, which may be coupled to the spacer.

In the inside of the bush 5, a coupling element 6 is designed, sealed in the intracorporal direction (in the schematic figure towards the top), indicated as a tapered ferrule (in the schematic on hand). The extracorporal coupling device 4 can be coupled to the coupling element 6.

As can be easily recognized, the bush 5 widens out significantly from its extracorporally oriented end 12 to its intracorporally oriented end 13 and comprises a smooth surface. The expansion from the extracorporal to the intracorporal end can occur in a linear fashion, but does not have to do so. In the present case, a slight curvature is represented.

In the represented exemplary embodiment, the ratio of the length of the bush 5 at its extracorporal facing end 12 to the length at the intracorporal facing end 13 is ca. 2.3:3.6=1:1.6. This ratio can lie within the interval between 1:1.2 and 1:2, in order to achieve a sufficient sealing effect of the atrophying surrounding (fatty) tissues. The surface here is smooth, so that no tissue of any kind can grow into the surface. On the other hand, no irritation is experienced due to friction effects, which would abet contamination.

Preferably, the surface of the bush has an antibacterial effect. For this purpose, it can be coated with silver or titanium.

The adaptation tube 7 reaches into the inside of the bush 5. The adaptation tube sits in the inside of the bush 6 in an interference fit and is removable, so that it can be removed for a necessary exchange and can be replaced by a new adaptation tube. The extracorporal coupling device 4 can be set into the adaptation tube 7. In the existing figure, the dimensions were so chosen, that, at the extracorporal coupling device 4 (when coupled on), the distal front edge 9 of the adaptation tube 7 is situated on the shoulder 10, which shoulder is formed on the coupling device 4, when the coupling device 4 is coupled to the spacer 3. By this means, a closed form is achieved, which prevents a contamination of the inside of the adaptation tube 7.

An osteosynthesis plate, formed on the implant 1, is represented. Preferably, the osteosynthesis plate attaches itself from the ventral side to the corticalis of the bone stump (not pictured). The open mesh, three-dimensional, spatial network structure 15, located on the inner side of the plate, adheres to the corticalis, so that the continuance of the implant 1 in the bone stump remains stable over the long term. The osteosynthesis plate 2 has the task of discharging forces arising at the transition from the implant shaft to its adapter socket 14 into the corticalis. The adapter socket 14 houses the plug cone 11 of the spacer 3.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

List of Reference Numbers

1 transcutaneous implant

2 osteosynthesis plate

3 spacer

4 extracorporal coupling device

5 bush

6 coupling element

7 adaptation tube

9 distal front edge

10 shoulder

11 plug cone

12 extracorporally oriented end of the bush

13 intracorporally oriented end of the bush

14 adapter socket

15 three-dimensional spatial network structure

1. A subcutaneous, intramuscular mounting for a rigid transcutaneous implant, which may be fixed intracorporally in a bone stump, comprising a spacer (3) between the implant and an extracorporal coupling device (4), which may be coupled thereto, whereby the spacer (3) is embodied as a rigid bush (5) with a coupling element (6), sealed in the intracorporal direction, to which the extracorporal coupling device (4) may be coupled, wherein,
the bush (5) widens out significantly from the end thereof facing the extracorporal direction (12) to the end thereof facing the intracorporal direction (13) and comprises a smooth surface.

2. A mounting according to claim 1, wherein the length of the base edge of the bush (5) at its extracorporally oriented end (12) as a ratio to the length at its intracorporally oriented end (13) as between 1:1.2 and 1:2.

3. A mounting according to claim 1, wherein the surface of the bush (5) has an antibacterial effect.

4. A mounting according to claim 3, wherein the surface of the bush (5) is plated with silver.

5. A mounting according to claim 3, wherein the surface of the bush (5) is plated with titanium.

6. A mounting according to claim 1 with an adaptation tube (7), which reaches into the inside of the bush (5), is seated in an interference fit, and is removable, to which adaptation tube the coupling device (4) can be coupled, which adaptation tube has an antibacterial effect, at least on its outer wall.

7. A mounting according to claim 6, wherein the adaptation tube (7) is constructed out of silver.

8. A mounting according to claim 6, wherein the adaptation tube (7) is constructed out of a material, whose outer wall is plated with silver.

9. A mounting according to claim 6, wherein the adaptation tube (7) is constructed out of a material, whose outer wall is coated with hydroxylapatite.

10. A mounting according to claim 6, wherein the adaptation tube (7) is constructed out of a material, whose outer wall is coated with calcium phosphate.

11. A mounting according to claim 6, wherein the adaptation tube (7) is constructed out of a material, whose outer wall is coated with titanium.

12. A mounting according to claim 6, wherein the adaptation tube (7) is constructed out of a material, whose outer wall is coated with plasma titanium spray.

13. A mounting according to claim 6, wherein the adaptation tube (7) is constructed out of polyurethane.

14. A mounting according to 6, wherein the adaptation tube (7) comprises such a length, that it is situated with its distal front edge (9) on a shoulder (10), which shoulder is formed on the coupling element (6) of the extracorporal coupling device (4), which is coupled thereto.
